(19) United States
(12) Patent Application Publication
(10) Pub. No.: US 2003/0157020 A1
(43) Pub. Date: Aug. 21, 2003

(54) Method of contrast enhanced magnetic resonance imaging and compounds useful therefor

(76) Inventors: Stefan Petersson, Malmo (SE); Ib Leunbach, Malmo (SE); Sven Mansson, Malmo (SE); Oskar Axelsson, Malmo (SE); Mikkel Thaning, Malmo (SE); Sven Andersson, Malmo (SE)

Correspondence Address: AMERSHAM HEALTH, IP DEPARTMENT, 101 CARNEGIE CENTER, PRINCETON, NJ, 08540-6231, US

(21) Appl. No.: 10/386,060

(22) Filed: Mar. 11, 2003

Related U.S. Application Data
(63) Continuation of application No. PCT/GB01/04085, filed on Sep. 12, 2001.
(60) Provisional application No. 60/256,995, filed on Jan. 5, 2001.

(30) Foreign Application Priority Data
Sep. 13, 2000 (NO) 20004561

(51) Int. Cl. A61K 49/00

(52) U.S. Cl. 424/9.3

(57) ABSTRACT

The present invention provides a method of contrast enhanced magnetic resonance imaging of a sample, said method comprising: a) administering a hyperpolarised MR contrast agent comprising non-zero nuclear spin nuclei into said sample for fluid dynamic investigations of the. vasculature, b) exposing said sample or part of the sample to radiation of a frequency selected to excite nuclear spin transitions in said non-zero nuclear spin nuclei, c) detecting MR signals from said sample using any suitable manipulation method including pulse sequences. The invention also provides novel compounds.

The present invention relates to methods of magnetic resonance imaging (MRI), in particular for use in MR angiography (MRA) and in fluid dynamic investigations of the vascular system and to the use therein of novel hyperpolarised contrast agents.

Magnetic resonance imaging is a diagnostic technique that has become particularly attractive to physicians as it is non-invasive and does not involve exposing the patient under study to potentially harmful radiation such as X-ray.

MR signal strength is dependent upon the population difference between the nuclear spin states of the imaging nuclei. In order to achieve effective contrast between MR images of different tissue types, it has long been known to administer to the subject MR contrast agents (e.g. paramagnetic metal species) which affect relaxation times in the zones in which they are administered or at which they congregate.

Contrast enhanced MRA is nowadays based on the injection of a paramagnetic contrast agent that shortens the relaxation times of the hydrogen atoms present in the blood vessels. By using imaging pulse sequences with short repetition times (TR) the background is suppressed. However the short T₂ relaxation leads to short acquisition time, high sampling rate and a reduced signal to noise ratio (SNR).

Angiography may also be performed using the “in-flow” technique without any contrast agent. This method also depends on the use of sequences utilizing short repetition times to suppress stationary spin present in the imaged volume. Consequently, it will result in a high sampling rate and a reduction of the SNR.

Both contrast enhanced MRA and the “in-flow” method may use the maximum intensity projection (MIP) software technique in order to generate angiograms. This methods makes it possible to generate projection images which mimic the x-ray way of creating angiograms. However, the quality of images generated using this method requires a high contrast to noise ratio (CNR) which may be difficult to achieve without disturbing artifacts due to insufficient suppression of the surrounding tissues.

The present invention thus relates in one aspect to a MRA method whereby the above-mentioned drawbacks are addressed. MRA measuring methods may thus be improved by using ex vivo nuclear spin polarisation and administration of nuclear spin polarised MR contrast agents. These agents comprise in their structure nuclei capable of emitting MR signals in a uniform magnetic field (e.g. ¹H, ¹³C, ¹⁵N, ¹⁹F, ²⁹Si and ³¹P nuclei) and capable of exhibiting a long T₁ relaxation time, and preferably additionally a long T₂ relaxation time.

Ex vivo methods have the advantage that it is possible to avoid administering the whole of, or substantially the whole of, the polarising agent to the sample under investigation, whilst still achieving the desired nuclear spin polarisation in the MR imaging agent. Thus such methods are less constrained by physiological factors such as the constraints imposed by the administrability, biodegradability and toxicity of agents in in vivo techniques.

When a hyperpolarised MR contrast agent is used, the background signal may, if the detection nucleus is not hydrogen, be totally absent. Thus it may be possible to use not only pulse sequences with short TR when an angiogram is collected. Instead sequences that make more efficient use of the available polarization, such as multi-echo sequences (e.g. RARE, EPI, GREASE), fully balanced gradient sequences (e.g. true FISP), steady state gradient sequences, and line scanning methods, may be utilized. An advantage of the present invention is that the extraction of micro-flow information is simplified.

Some of the advantages with the present invention for magnetic resonance angiography (MRA) using hyperpolarised contrast agents are as follows:

images can be obtained without any background signal,

there is no need for pulse sequence techniques to suppress stationary spins,

projection images showing the blood vessels in a arbitrary direction,

High SNR to allow for coronary angiography, and

due to long T₁ relaxation values, vessels far from the injection point may be enhanced.

Techniques have been developed which involve ex vivo nuclear spin polarisation of contrast agents containing non-zero nuclear spin nuclei (e g. ³He), prior to administration and MR signal measurement.

It has also been demonstrated that it is possible to hyperpolarise compounds comprising e.g. ¹³C and ¹⁵N ex vivo, in order to produce injectable polarised contrast agents e.g. by polarisation transfer from a noble gas, by “brute force”, by the dynamic nuclear polarisation (DNP) or the para-hydrogen methods (see, for example, the present Applicant's publications WO99/35508 and WO 99/24080, the disclosures of which are hereby incorporated by reference). Some of these techniques involve the use of polarising transfer agents which are defined as any agents suitable for producing the ex vivo polarisation of an MR contrast agent.

In all aspects of the present invention, any suitable way of hyperpolarisation may be used. In effect, it is not dependent on the hyperpolarisation method used. However, in many situations hyperpolarisation methods using para-hydrogen and DNP are preferred.

After the ex vivo hyperpolarisation step is performed, any polarising transfer agent is preferably separated from the composition comprising the polarised MR contrast agent. The polarised MR contrast agent is then administered to the body using any suitable delivery system and injected into the patient for an angiographic and/or fluid dynamic investigation of the vascular system. The present invention thus relates in one aspect to a method of contrast enhanced magnetic resonance imaging of a sample, preferably a human or non-human animal body, said method comprising:

a) administering, e.g. by injection, a hyperpolarised MR contrast agent comprising non-zero nuclear spin nuclei into said sample for angiographic investigations,

b) exposing said sample or part of said sample to radiation of a frequency selected to excite nuclear spin transitions in said non-zero nuclear spin nuclei,

c) detecting MR signals from said sample using any suitable manipulation method including pulse sequences,

d) optionally ensuring that the execution of the pulse sequence and/or the administration of the contrast agent are gated against heart rhythm and/or the respiration rhythm of the body,

e) optionally, generating an image, spectroscopic data, dynamic flow data or physiological data from said detected signals.

In some investigations and according to a preferred aspect of the present invention with zero background signal, angiograms may be generated by using projection in the desired direction of the vessels in question. The lack of a background signal reduces the risk of “back folding” artifacts. This may be particularly useful when coronary angiography is performed which is another preferred aspect of the invention. An image of a slice of the same thickness as the heart, in any given direction, may be used to generate a projection of the complete heart. This approach mimics the way X-ray angiography is performed.

In conventional fluid dynamic methods for investigations of the vascular system used today e.g. for micro-flow (perfusion), methods are based on recording signal drop during the passage of a contrast bolus or by using tagging methods. The tagging methods use the inflow of blood, from the tagged region, to the imaged region and measure the change in signal intensity as the base for calculation of a perfusion map. Such methods generate perfusion maps and regional cerebral blood volume (rCBV) maps with only limited SNR.

In the case of conventional velocity measurements, the methods are based on signal phase data and the signal medium is either blood or blood comprising a paramagnetic contrast medium e.g. a Gd-based contrast agent. However, such velocity measurement using phase methods are sensitive to phase error due to the surrounding tissues.

When a hyperpolarised MR contrast agent is used in a method as provided by the present invention, the background signal may, if the detection nucleus is not hydrogen, be totally absent. Thus it may be possible to use pulse sequences other than those with short TR. Instead sequences that more efficient make use of the available polarization, such as multi echo sequences (RARE, EPI, GREASE), fully-balanced gradient sequences (e.g. true FISP), steady state gradient sequences, and line scanning methods, may be utilized. An advantage with the present invention is that the extraction of micro-flow information is simplified.

Thus viewed from another aspect, the invention provides a fluid dynamic investigation of the vascular system whereby the above-mentioned drawbacks are addressed. Methods for obtaining flow and micro-flow measurements and/or quantifying data are preferred. Especially preferred are methods for obtaining perfusion, flow velocity, flow profile, tissue perfusion maps and regional blood volume including regional cerebral blood volume (rCBV) data.

The present invention thus relates in another aspect to a method of contrast enhanced magnetic resonance imaging of a sample, preferably a human or non-human animal body, said method comprising:

a) administering, e.g. by injection, a hyperpolarised MR contrast agent comprising non-zero nuclear spin nuclei into said sample for fluid dynamic investigations of the vasculature,

b) exposing said sample or part of said sample to radiation of a frequency selected to excite nuclear spin transitions in said non-zero nuclear spin nuclei,

c) detecting MR signals from said sample using any suitable manipulation method including pulse sequences,

d) optionally ensuring that the execution of the pulse sequence and/or the administration of the contrast agent are gated against heart rhythm and/or the respiration rhythm of the body,

e) optionally, generating an image, spectroscopic data, dynamic flow data, perfusion data, blood volume data and/or any other suitable physiological data from said detected signals.

According to a preferred embodiment of the present invention, the specific pulse sequence used will depend on the flow velocity in the vessel type to be imaged. In some situations, fast, single shot sequences (e.g. EPI, RARE, GREASE, BURST, QUEST) are preferred for imaging of the coronary arteries.

Any diffusion of the hyperpolarised contrast agent molecule may be measured using the method suggested by Stajskal et al and referred to as the Stajskal-Tanner (ST) method in standard NMR and MRI literature. The ST sequence works by the dephasing and subsequent rephasing of protons using two equally-sized gradient pulses separated by a 180° pulse. This gradient/rf pulse sequence may be incorporated as a pre-phase before the actual data collection part of a pulse sequence. Several different pulse sequences (e.g. spin echo, EPI, STEAM, RARE) have been modified in order to incorporate the ST-method. During the application of the ST part of a diffusion sequence the protons NMR-signal is attenuated due to T₂ relaxation. The effective TE (echo time) may often reach values of 60 ms or longer. The influence from relaxation may thus be strong. This relaxation will result in signal attenuation and a reduced SNR. When a hyperpolarised contrast medium with a long T₁/ T₂ is used, the signal attenuation will be less due to relaxation, when utilising a pulse sequence with a long TE.

The lack of background signal also simplifies the calculation of micro-flow data as perfusion maps and regional cerebral blood volume (rCBV) maps. This method is thus a preferred aspect of the invention.

Due to the long T₁ relaxation time of the hyperpolarised contrast agent, vessels far from the injection point may be visualized, including vessels in the brain and in the lungs, and this is another preferted aspect of the invention.

As mentioned earlier as an optional step (step d) of this invention, and in order to optimize the image window used for angiography or for fluid dynamic investigations of the vasculature, the execution of the pulse sequence and/or the administration, e.g. injection, of the hyperpolarised contrast agent may need to be gated against the heart and/or respiration of the patient. The gating may also be used to ensure that the organ/imaged volume is in the same position during the collection of the series of images. The gating step may be performed in order to image the volume/organ in question before and during the passage of a contrast medium bolus.

In all aspects of the invention, it is preferred to use a tagging or saturation technique. This technique may be used to show only those hyperpolarised spins in the final image that have entered the imaged region through specific vessels or from a given flow direction. It may also be used to remove the signal from hyperpolarised spins in a given part of an imaged volume, e.g. within the heart when the coronary arteries are to be visualized.

Tagging and saturation techniques may preferably be used when micro-flow/perfusion data is collected. This technique may be performed by destroying all of the hyperpolarisation, using a saturation pulse from the volume to be investigated and by observing the inflow due to micro-flow. The observation is then performed using a volume selective image pulse sequence. Any inflow into a small volume element (voxel) may also be measured using a point scanning method. The performed measurements may include collection of spectroscopic and/or physiological information in order to distinguish between different tissue types or/and flow velocities.

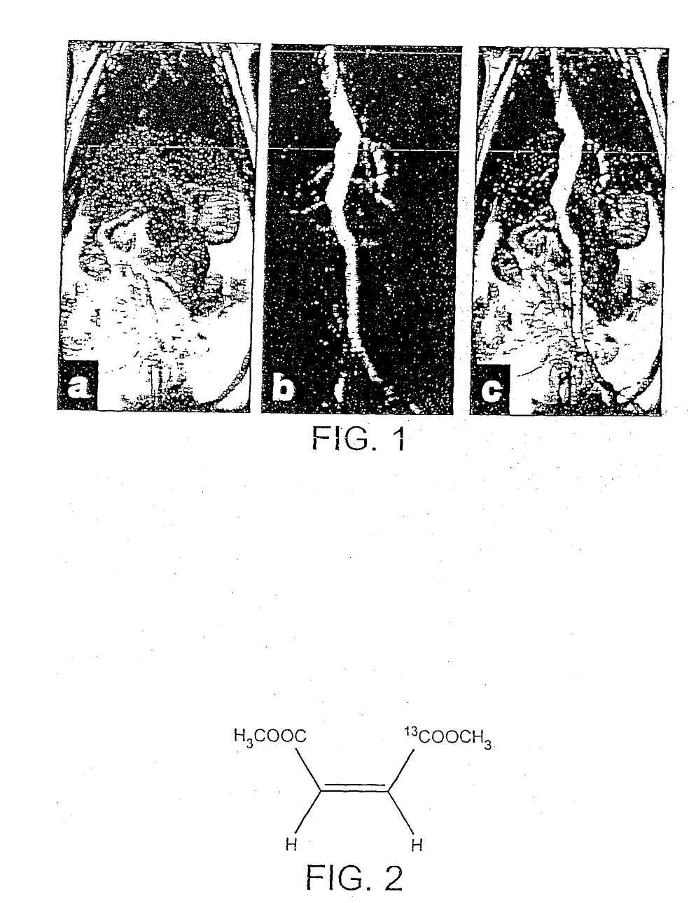

In another preferred aspect of the invention, a “native image” of the body (i.e. one obtained prior to administration of the hyperpolarised MR contrast agent or one obtained for the administered MR contrast agent without prior polarisation as in a conventional MR experiment) may be generated to provide structural (e.g. anatomical) information upon which the image obtained in the method according to the invention may be superimposed. A “native image” is generally not available where ¹³C or ¹⁵N is the imaging nucleus because of the low abundance of ¹³C and ¹⁵N in the body. In this case, a proton MR image may be taken to provide the anatomical information upon which the ¹³C or ¹⁵N image may be superimposed, see e.g. FIG. 1c of the accompanying drawings.

Using standard phase contrast techniques and/or extra gradient/rf pulse to perform encoding, of spatial or movement information, flow velocity may be measured. Also the flow velocity profile may be measured using through-plane sequences.

By “angiography”, we mean any investigation regarding any angiographic vessel, i.e. the arteries and the capillary system. In some situations, measurements of veins may also be covered by the present invention. A preferred aspect of the invention provides MRA imaging of the arteries.

By the “vascular system”, we mean any system of blood containing vessels, i.e. arteries, veins and capillaries.

By “hyperpolarised”, we mean polarised to a level over that found at room temperature and IT, preferably polarised to a polarisation degree in excess of 0.1%, more preferably in excess of 1%, even more preferably in excess of 10%.

The hyperpolarised contrast agent should preferably exhibit a long T₂ relaxation time, preferably greater than 0.5 secs, more preferably greater than 1 sec, even more preferably than 5 secs. Suitable MR imaging agents according to the invention, may contain nuclei such as e.g. ³Li, ¹³C, ¹⁵N, ¹⁹F, ²⁹Si or ³¹P, as well as ¹H, preferably ¹H, ¹³C, ¹⁵N, ¹⁹F and ³¹P nuclei, with ¹H, ¹³C, ¹⁵N and ³¹P nuclei being particularly preferred. Most especially preferred are ¹³C nuclei.

As noted above, ¹H, ¹³C, ¹⁵N and ³¹P are the nuclei most suited to use in a method of the present invention with ¹³C being most especially preferred. ¹H nuclei have the advantages of being present in high concentration in natural abundance and having the highest sensitivity of all nuclei. ¹³C nuclei are advantageous as the background signal from hyperpolarised ¹³C nuclei is very low and much less than from, for example, ¹H nuclei. ¹⁹F nuclei have the advantage of high sensitivity. Hyperpolarisation of contrast agents comprising ³¹P nuclei allows endogenous substances to be used.

Where the MR imaging nucleus is other than a proton (e.g. ¹³C or ¹⁵N), there will be essentially no interference from background signals (the natural abundance of ¹³C and ¹⁵N, for instance, being negligible) and the image contrast will be advantageously high. This is especially true where the MR contrast agent itself is enriched above natural abundance in the MR imaging nucleus. Thus the method according to the invention has the benefit of being able to provide significant spatial weighting to a generated image.

The MR contrast agent should preferably be artificially enriched with nuclei (e.g. 15N and/or ¹³C nuclei) having a long T, relaxation time.

The long T₁ relaxation time of certain ¹³C and ¹⁵N nuclei is particularly advantageous and certain MR contrast agents containing ¹³C or ¹⁵N are therefore preferred for use in the present method. Preferably the polarised MR contrast agent has an effective nuclei ¹³C polarisation of more than 0.1%, more preferably more than 1.0%, even more preferably more than 10%, particularly preferably more than 25%, especially particularly preferably more than 50% and finally most preferably more than 95%.

The MR contrast agent is more preferably ¹³C enriched at carbonyl or quaternary carbon positions, given that a ¹³C nucleus in a carbonyl group or in certain quaternary carbons may have a T₁ relaxation time typically of more than 2s, preferably more than 5s, especially preferably more than 30 s. Preferably the ¹³C enriched compound should be deuterium labelled, especially adjacent the ¹³C nucleus. Preferred ¹³C enriched compounds are those in which the ¹³C nuclei are surrounded by one or more non-MR active nuclei such as O, S, C or a double or triple bond. MR contrast agents for use in methods of the present invention are of the formula (I):

CX₄ (I)

wherein each X is independently D, CD₃, CD₂OR′, SO₃H, SO₂H, SO₂NH₂, CONR′₂, CO₂H and OCHO,

wherein R¹ is independently H or Me,

or two of the X groups and the C atom they are attached to form either the 3-membered ring

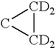

or the 4-membered ring

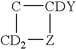

wherein Y is D or CD₂OR¹

and Z is CD₂, CD(CD₂OR¹) or O.

Shown below as compounds 1-17 are particular examples of agents suitable for use in the present invention. Such agents are water soluble, non- toxic, easy to synthesize and have relatively long T₁-values in water, for example in excess of 60 secs.

For instance, compounds 1 and 2 are found to have T₁ values of 95 secs and 133 secs, respectively.

With the exception of compounds 1-3 shown below which are known from the applicant's own published application no. WO-A-99/35508, these agents are themselves novel and form a further aspect of the present invention. Examples are shown below as compounds 4-17. The agents can be ¹³C enriched.

Viewed from a further aspect the invention provides a physiologically tolerable MR imaging agent composition comprising an MR imaging agent together with one or more physiological tolerable carriers or excipients, said imaging agent being chosen from one of the compounds in general formula (I) above, preferably compounds numbered 1-17 as below, for example compounds numbered 4-17 as below.

Viewed from a still further aspect the invention provides the use of a compound from general formula (I) above, preferably a compound numbered 1-17 as below, for example a compound 4-17 as below, in a method of the present invention.

Viewed from a yet still further aspect the invention provides the use of a compound from general formula (I) above, preferably a compound numbered 1-17 as below, for example a compound 4-17 as below, for the manufacture of an MR imaging agent for use in a method of diagnosis involving the generation of an MR image by MR imaging of a human or non-human being.

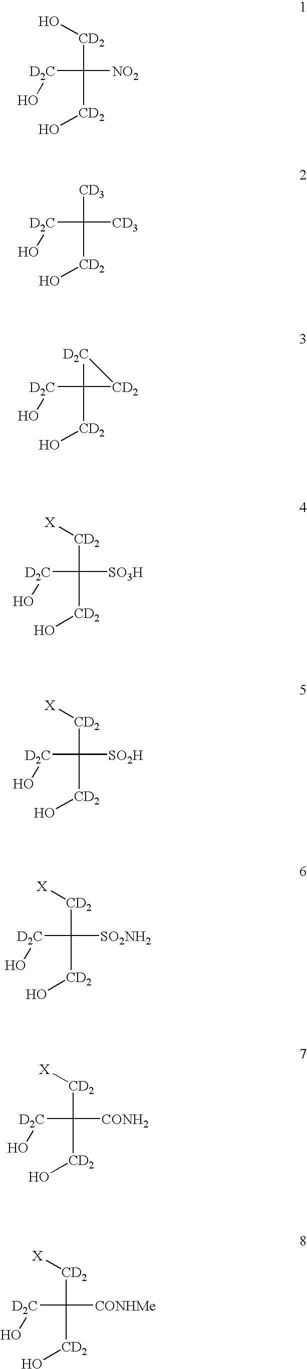

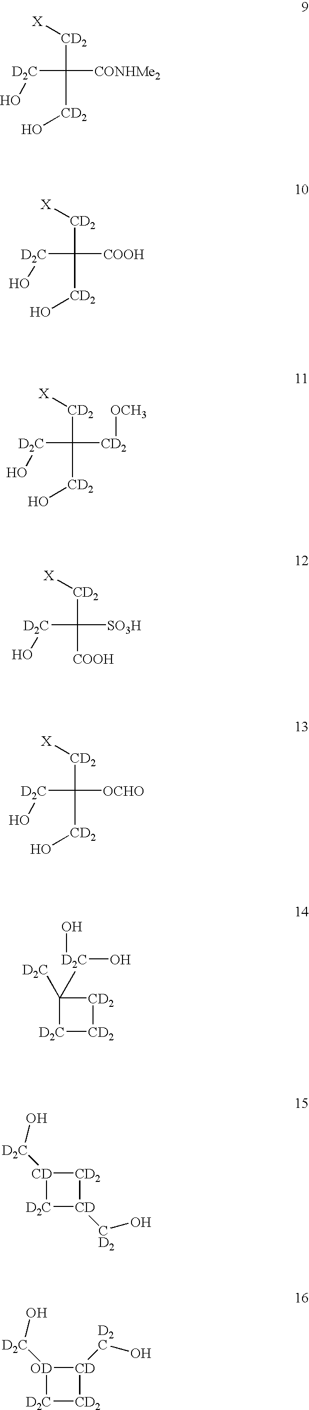

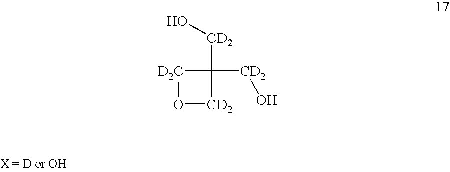

The MR contrast agent should of course be physiologically tolerable or be capable of being provided in a physiologically tolerable, administrable form with conventional pharmaceutical or veterinary carriers or excipients. Preferred MR contrast agents are soluble in aqueous media (e.g. water) and are of course non-toxic.

The formulation, which preferably will be substantially isotonic, may conveniently be administered at a concentration sufficient to yield a 1 micromolar to 10M concentration of the MR contrast agent in the imaging zone; however the precise concentration and dosage will of course depend upon a range of factors such as toxicity and the administration route.

Parenterally administrable forms should of course be sterile and free from physiologically unacceptable agents, and should have low osmolality to minimize irritation or other adverse effects upon administration and thus the formulation should preferably be isotonic or slightly hypertonic.

It may be convenient to inject simultaneously at a series of administration sites such that a greater proportion of the vascular tree may be visualized before the polarization is lost through relaxation.

The dosages of the MR contrast agent used according to the method of the present invention will vary according to the precise nature of the MR contrast agents used and of the measuring apparatus. Preferably the dosage should be kept as low as possible while still achieving a detectable contrast effect. In general, the maximum dosage will depend on toxicity constraints.

After the polarisation, the hyperpolarised MR contrast agent may be stored at low temperature e.g. in frozen form. Generally speaking, at low temperature the polarisation is retained longer and thus polarised contrast agents may conveniently be stored e.g. in liquid nitrogen. Prior to administration, the MR contrast agent may be rapidly warmed to physiological temperatures using conventional techniques such as infrared or microwave radiation.

The contents of all publications referred to herein are incorporated by reference. Embodiments of the invention are described further with reference to the following non-limiting Examples and the accompanying drawings.

### EXAMPLE 1

The method of para-hydrogen polarisation transfer as described in WO 99/24080 (to Nycomed Imaging AS) using a (PPh₃)RhCl catalyst was performed using a maleic acid dimethyl ester C labelled in the carbonyl group, (see FIG. 2 of the accompanying drawings). After polarisation, the polarised compound was injected as a contrast medium into the tail vein of a rat.

The concentration and the polarization of ¹³C nuclei in the bolus that was injected into the rat was 150 mM and approximately 0.3%, respectively, and the imaging was performed, see FIG. 1 of the accompanying drawings.

The images shown in FIG. 1 were generated using a BioMed animal scanner operating at 2.4 Tesla. The image shown in FIG. 1a is a proton image and has been generated using a standard spin echo pulse sequence and without the use of any contrast medium. Pulse sequence parameters were TR/TE/α=3.3 ms/1.4 ms/5° and a total scan time of 4:23 min. A dose of the hyperpolarised contrast medium was then generated. The resonance frequency was changed to the one needed to perform ¹³C imaging and a single shot PARE sequence was executed. The to total scan time was 0.9 sec., the used inter-echo time was 28 ms and the matrix size was 128×32. The resulting image is shown in FIG. 1b. The total lack of background signal is clearly demonstrated. This image was generated as a projection right through the complete animal demonstrating the possibility of generating an angiogram in the same way that when x-rays are used. In FIG. 1c the ¹³C image has been superimposed on the hydrogen image.

1. A method of contrast enhanced magnetic resonance imaging of a sample, said method comprising:
a) administering a hyperpolarised MR contrast agent comprising non-zero nuclear spin nuclei into said sample for fluid dynamic investigations of the vasculature,
b) exposing said sample or part of the sample to radiation of a frequency selected to excite nuclear spin transitions in said non-zero nuclear spin nuclei,
c) detecting, MR signals from said sample using any suitable manipulation method including pulse sequences,
d) optionally ensuring that the execution of the pulse sequence and/or the administration of the contrast agent are gated against heart rhythm and/or the respiration rhythm of the body,
e) optionally, generating an image, spectroscopic data, dynamic flow data, perfusion data, blood volume data and/or any other suitable physiological data from said detected signals.

2. The method of claim 1 wherein said fluid dynamic investigation of the vasculature comprises angiographic investigations.

3. The method of claim 1 wherein said data is obtained using the Stajskal-Tanner method.

4. The method as claimed in any one of the preceding claims further comprising use of a tagging or saturation technique.

5. The method as claimed in any one of the preceding claims wherein said non-zero nuclear spin nuclei is selected from the group consisting of ¹H ³Li, C, 1N, F, S i and ³¹P.

6. The method as claimed in any one of the preceding claims wherein said non-zero nuclear spin nuclei is selected from the group consisting of ¹H, ¹³C, ¹⁵N, and ³¹P, preferable wherein said nuclei are ¹³C nuclei.

7. The method as claimed in claim 6 wherein the MR contrast agent has an effective nuclei ¹³C polarisation of more than 1% preferably more than 95%.

8. The method as claimed in claim 6 wherein the MR contrast agent is ¹³C enriched at carbonyl or quaternary carbon positions.

9. The method as claimed in claim 8 wherein said ¹³C enriched compound is deuterium labelled adjacent said ¹³C nucleus.

10. The method as claimed in any one of claims 6 to 9 wherein said ¹³C nuclei are surrounded by one or more non-active nuclei or entities selected from the group consisting of 0, S, C or a double or triple bond.

11. A compound of formula (I):
CX₄ (I)
wherein each X is independently D, CD₃, CD₂OR′, SO₃H, SO₂H, SO₂NH₂, CONR′₂, CO₂H and OCHO,
wherein R¹ is independently H or Me,
or two of the X groups and the C atom they are attached to form either the 3-membered ring 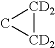
or the 4-membered ring 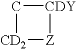
wherein Y is D or CD₂OR¹
and Z is CD₂, CD (CD₂OR¹) or O,
with the proviso that the compound is not one of the following:

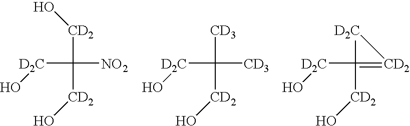

12. A compound as claimed in claim 11 selected from the following:

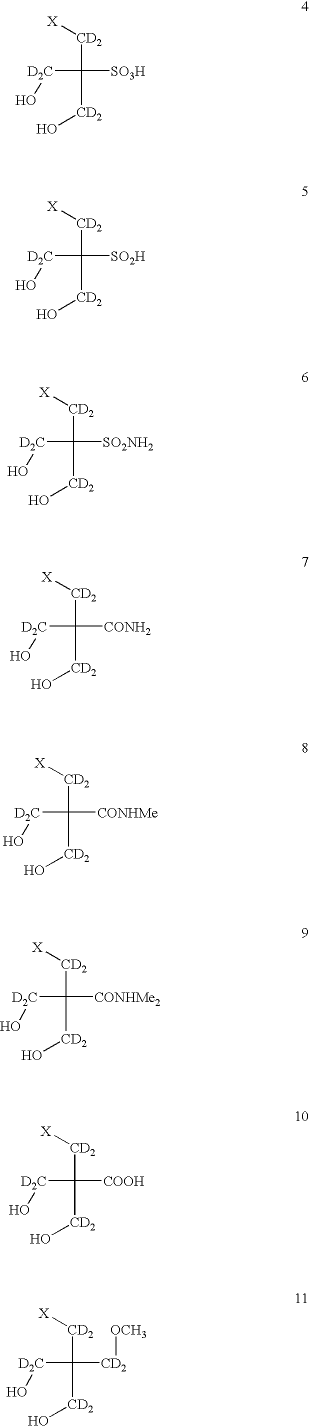

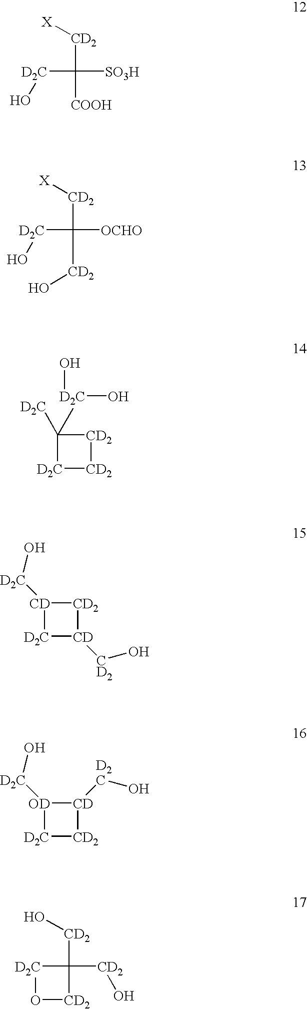

13. Use of a compound of the formula (I):
CX₄ (I)
wherein each X is independently D, CD₃, CD₂OR′, SO₃H, SO₂H, SO₂NH₂, CONR′₂, CO₂H and OCHO,
wherein R¹ is independently H or Me,
or two of the X groups and the C atom they are attached to form either the 3-membered ring 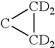
or the 4-membered ring 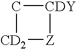
wherein Y is D or CD₂OR¹
and Z is CD₂, CD(CD₂OR¹) or O,
in a method as claimed in any one of claims 1 to 10.

14. Use of a compound of the formula (I) as defined in claim 13 for the manufacture of an MR imaging agent for use in a method of diagnosis involving the generation of an MR image by MR imaging of a human or non-human being.

15. A physiologically tolerable MR imaging agent composition comprising an MR imaging agent together with one or more physiological tolerable carriers or excipients, said imaging agent comprising a compound of the formula (I) as defined in claim 13.
